(19) United States
(12) Patent Application Publication
(10) Pub. No.: US 2024/0423958 A1
(43) Pub. Date: Dec. 26, 2024

(54) C-7 FUNCTIONALIZED ARTEMISININ DERIVATIVES FOR THE TREATMENT OF MALARIA

(71) Applicant: University of the Incarnate Word, San Antonio, TX (US)

(72) Inventors: Paulo Carvalho, San Antonio, TX (US); Karine Le Roch, San Antonio, TX (US); Mitchell Avery, San Antonio, TX (US); John Williamson, San Antonio, TX (US); Amar Chittiboyina, San Antonio, TX (US); Matthew Valdez, San Antonio, TX (US); Zeinab Chahine, San Antonio, TX (US); Melinda Baldwin, San Antonio, TX (US)

(21) Appl. No.: 18/743,985

(22) Filed: Jun. 14, 2024

Related U.S. Application Data
(60) Provisional application No. 63/508,038, filed on Jun. 14, 2023.

(51) Int. Cl. A61K 31/4178; A61K 31/357; A61K 31/366; A61P 33/06

(52) U.S. Cl. CPC A61K 31/4178; A61K 31/357; A61K 31/366; A61P 33/06

(57) ABSTRACT

C-7 functionalized artemisinin derivatives, and methods of producing and using C-7 functionalized artemisinin derivatives, for the treatment of malaria. Semi-synthesis of artemisinin analogs based on the functionalization of C-7 through fungal biotransformation. Fungal fermentation techniques were used to add hydroxyl groups to artemisinin positions that are difficult to access chemical, namely C-7.

### STATEMENT REGARDING FEDERALLY FUNDED RESEARCH

This invention was made with government support, grant SC3GM136576, awarded by the National Institutes of Health. The government has certain rights in the invention.

The present inventions are compositions and methods for treating parasitic infections using C-7 functionalized artemisinin derivatives.

### FIELD OF INVENTION

The present inventions relate generally to the field of medicine and concerns novel artemisinin derivatives and their pharmaceutical compositions, and methods for treating parasitic infections.

### BACKGROUND

Malaria impacts about 3.3 billion people annually, (World Health Organization 2012) causing over half a million people deaths, the majority being children under the age of five. Widespread cases of malaria continue to plague the poorest and most underdeveloped corners of the world, predominantly in sub-Saharan Africa, Southeast Asia, and South America. Of the 14 subgenera of Plasmodium only five species can infect humans. Plasmodium falciparum is responsible for roughly 50% of all cases and most fatalities caused by malaria.

According to the Center for Disease Control, once the human blood stage begins, parasites go through a cycle of developmental stages called ring, trophozoite, and schizont. Resistance to most current therapies such as chloroquine has been well documented. A recent trend, not yet characterized as full resistance, has been identified in P. falciparum strains isolated in South Asia where the parasite can resume infection after treatment with current artemisinin therapies. The definition of partial resistance of the parasite to artemisinin, is slow or delayed parasite clearance. This has been attributed to the parasite's ability to remain in an immature trophozoite state during its human blood stage. As a result of the short half-life of these therapies, the parasite can evade the harmful effects of the drugs in its early erythrocytic cycle when it is not vulnerable to the currently approved artemisinin-based therapies.

In the case of artemisinin, analogs studied thus far have consisted mainly of compounds with substituents at C-9, C-10, C-11. The medicinal chemistry community has largely focused on preparing derivatives of artemisinin with modifications at the C-10 position. Many C-10 modified artemisinin analogs have better activity relative to their parent compound, however these derivatives fail to adequately address metabolic and stability issues in vivo. The metabolic issues associated with artemisinin and its derivatives are attributed in part to hydroxylation (and subsequent glycosylation) at the C-6 or C-7 position, thus allowing for rapid rates of clearance and excretion in vivo. There are very few examples in the literature of artemisinin derivatives with substituents at C-3 through C-8, given the difficulty involved in the incorporation of functional groups by usual synthetic operations in those positions of the carbocycle. A limited number of C-7 derivatives have been disclosed in several prior structure activity relationship studies which sought to improve the poor pharmacokinetic properties associated with artemisinin. Fluorine atom(s) were incorporated at the C-6 and C-7 positions, these fluorinated analogs possess only slightly enhanced anti-malarial activity (between one and two-fold) relative to the current artemisinin therapies. Analogs bearing glucuronide conjugates at the C-7 position have also been prepared and examined for their antimalarial activity, however these analogs did not display activity superior to currently used artemisinin-based therapies. These studies suggest that simply modifying artemisinin at distal sites such as C-6 or C-7 does not intrinsically impart enhanced biological activity to artemisinin analogs.

Given the current limitations associated with approved artemisinin based therapies such as poor bioavailability and the emerging number of drug resistant cases, the need to develop novel small molecules with greatly enhanced activity to treat malaria is warranted.

### SUMMARY OF THE INVENTION

To address the alarming number of artemisinin resistant cases reported in medical literature, the present invention uses semi-synthesis of artemisinin analogs based on the functionalization of C-7 through fungal biotransformation. In other embodiments, a photochemical preparation of the staring material could be used instead of fungal biotransformation. This invention is directed toward compounds of Formula A and B, or pharmaceutically acceptable salts thereof, and methods of their use. Fungal fermentation techniques were used to add hydroxyl groups to artemisinin positions that are difficult to access chemical, namely C7.

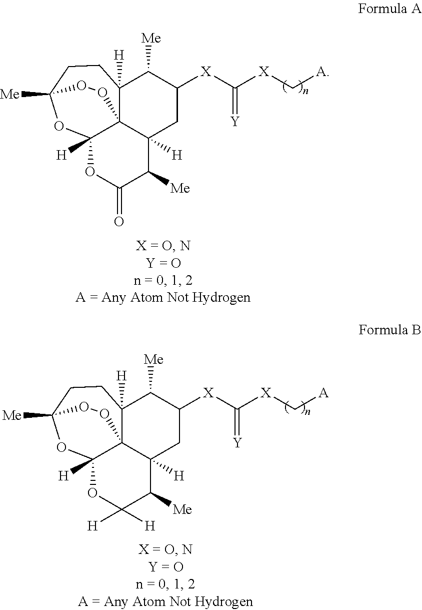

The biological activity of C-7 functionalized artemisinin analogs is described with reference to Paragraphs [0010]-[0012].

Plasmodium falciparum Culture

Asexual parasite Culture and maintenance—P. falciparum 3D7 (MRA-102) parasites were propagated in 5% of human O+ erythrocytes and 10 mL of RPMI-1640 medium containing 0.5% Albumax II (Invitrogen), 2 mM L-glutamine, 50 mg/L hypoxanthine, 25 mM HEPES, 0.225% NaHCO3 and 10 mg/mL gentamycin. They were maintained at 37° C. and gassed with a sterile mixture of 5% O2, 5% CO2 and 90% N2 A,B. Parasite synchronization was achieved using two 5% D-sorbitol treatments 8 hours apart C.

### SYBR Green Fluorescent Based Drug Assay

The anti-malarial activity of individual analogs were evaluated in-vitro against P. falciparum 3D7 (MRA-102, drug sensitive) and W2 (MRA-157, Chloroquine resistant) strains (ATCC® Manassas, VA) via SYBR green-I based fluorescence assay. Parasite growth rate and stages of development were determined via microscopy through Giemsa stained smears of the cultures. Serial dilutions of the compounds were prepared in 96-well plates (Corning, Costar 3904) followed by the introduction of synchronized ring stage parasites cultures at 0.5% parasitemia and 2.5% hematocrit added to each well. Eight wells were treated with vehicle control (DMSO) as positive control. Plates were incubated in a modular incubation chamber (Billups-Rothenberg, Del Mar, CA) maintained at 37° C. for 72 h in a low oxygen environment (96% N2, 3% CO2, 1% O2). After 72 h, plates were removed from incubation and stored at −80° C. for 24 hr. Plates were subsequently thawed followed by incubation with 1× volume of lytic buffer (20 mM Tris-HCl, pH 7.5, 5 mM EDTA, 0.008% Saponin, 0.08% Triton X-100) containing SYBR Green 1× for 4-6 h at 37° C. in darkness. Plates were read with a Molecular Devices SpectraMAX Gemini EM at Ex. 495 nm, Em. 525 nm. Assessment of anti-malarial activity of compounds was made on the basis of fifty percent inhibitory concentration values (IC₅₀) determined by DNA content of the parasite (SigmaPlot 10 (Systat).

Representative examples of the antimalarial activity of the compounds presented in this invention are shown in Table 1 and Table 2.

### SUMMARY OF THE METHODOLOGY

7β-hydroxy-artemisinin was obtained using a modified procedure found in the literature. (Parshikov, Muraleedharan et al 2004). 7β-hydroxy-10-deoxoartemisinin was obtained using a modified procedure found in the literature. (Parshikov, Muraleedharan et al 2004).

A representative procedure used to prepare carbonates was to a solution of 7β-hydroxy-artemisinin (0.10 mmol) and 2,6-lutidine (0.40 mmol) in dry dichloromethane (0.75 ml) was added a solution of the appropriate chloroformate (0.2 mmol) in dry dichloromethane (0.5 ml) drop-wise, and the resulting solution was stirred at room temperature for 24 h. The solvents were evaporated under reduced pressure and the crude residue was purified by flash column chromatography on silica gel using ethyl acetate-hexanes as the eluent to afford the desired carbonates in 45-80% yields.

This representative procedure for the preparation of carbonates is further shown in the following diagram: procedure is shown in the following diagram:

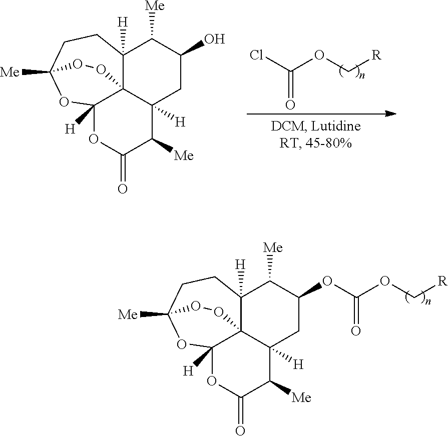

Embodiments of C-7 functionalized artemisinin derivative carbonates prepared are described and shown in paragraphs [0017]-[0037].

### Methyl (3R,5aS,6S,7S,8aS,9R,12S,12aR)-3,6,9-trimethyl-10-oxodecahydro-3,12-epoxy[1,2]dioxepino[4,3-i]isochromen-7-yl-carbonate (1)

¹H NMR (CDCl₃): δ 1.04 (d, 3H, J=6.0 Hz), 1.20 (d, 3H, J=7.3 Hz), 1.27 (m, 1H), 1.44 (s, 3H), 1.50-1.58 (m, 2H), 1.59-1.66 (m, 1H), 1.89-2.12 (m, 3H), 2.25 (dt, 1H, J=4.4, 13.0 Hz), 2.38-2.49 (m, 1H), 3.33-3.42 (m, 1H), 3.78 (s, 3H), 4.31 (dt, 1H, J=4.6, 10.7 Hz), 5.90 (s, 1H); ¹³C NMR (CDCl₃): δ 12.51, 15.30, 24.70, 25.12, 28.40, 32.49, 35.66, 41.37, 41.84, 47.81, 54.93, 78.27, 78.87, 93.11, 105.48, 155.33, 171.13; ESI-MS m/z [M+Na]⁺C₁₇H₂₅O₈Na:

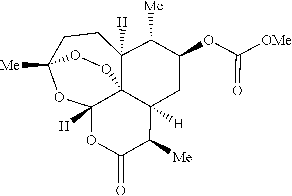 [SMILES: COC(=O)O[C@H]1C[C@H]2[C@@H](C)C(=O)O[C@@H]3O[C@@]4(C)CC[C@@H]([C@@H]1C)[C@]32OO4]

### Ethyl(3R,5aS,6S,7S,8aS,9R,12S,12aR)-3,6,9-trimethyl-10-oxodecahydro-3,12-epoxy[1,2]dioxepino[4,3-i]isochromen-7-yl-carbonate (2)

¹H NMR (CDCl₃): δ 1.02 (d, 3H, J=6.0 Hz), 1.21 (d, 3H, J=7.2 Hz), 1.26 (m, 1H), 1.31 (t, 3H, J=7.2 Hz), 1.45 (s, 3H), 1.51 (m, 1H), 1.57 (m, 1H), 1.65 (m, 1H), 1.97 (m, 1H), 2.01 (m, 1H), 2.09 (m, 1H), 2.25 (dt, 1H, J=4.4, 12.8 Hz), 2.46 (m, 1H), 3.38 (m, 1H), 4.20 (q, 2H, J=7.2 Hz), 4.31 (dt, 1H, J=4.4, 10.4 Hz), 5.92 (s, 1H); ¹³C NMR (CDCl₃): δ 12.45, 14.10, 15.26, 24.61, 25.05, 28.33, 32.42, 35.56, 41.30, 41.70, 47.67, 64.21, 78.23, 78.46, 93.07, 105.43, 154.62, 171.20; ESI-MS m/z [M+H]⁺C₁₈H₂₇O₈: 371.2.

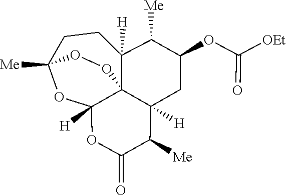 [SMILES: CCOC(=O)O[C@H]1C[C@H]2[C@@H](C)C(=O)O[C@@H]3O[C@@]4(C)CC[C@@H]([C@@H]1C)[C@]32OO4]

### 1-Propyl(3R,5aS,6S,7S,8aS,9R,12S,12aR)-3,6,9-trimethyl-10-oxodecahydro-3,12-epoxy[1,2]dioxepino[4,3-i]isochromen-7-yl-carbonate (3)

¹H NMR (CDCl₃): δ 0.95 (t, 3H, J=7.2 Hz), 1.04 (d, 3H, J=5.6 Hz), 1.20 (d, 3H, J=6.8 Hz), 1.26 (m, 1H), 1.44 (s, 3H), 1.49 (m, 1H), 1.55 (m, 2H), 1.63 (m, 1H), 1.70 (m, 1H), 1.96 (m, 1H), 1.98 (m, 1H), 2.07 (m, 1H), 2.24 (m, 1H), 2.45 (m, 1H), 3.37 (m, 1H), 4.09 (t, 2H, J=6.8 Hz), 4.31 (dt, 1H, J=4.4, 10.4 Hz), 5.90 (s, 1H); ¹³C NMR (CDCl₃): δ 10.12, 12.51, 15.31, 21.94, 24.70, 25.11, 28.38, 32.50, 35.65, 41.37, 41.79, 47.76, 69.80, 78.33, 78.54, 93.14, 105.46, 154.83, 171.19; ESI-MS m/z [M+H]⁺C₁₉H₂₉O₈Na: 385.2.

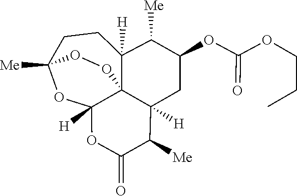 [SMILES: CCCOC(=O)O[C@H]1C[C@H]2[C@@H](C)C(=O)O[C@@H]3O[C@@]4(C)CC[C@@H]([C@@H]1C)[C@]32OO4]

### 2-Methylpropyl(3R,5aS,6S,7S,8aS,9R,12S,12aR)-3,6,9-trimethyl-10-oxodecahydro-3,12-epoxy[1,2]dioxepino[4,3-i]isochromen-7-yl-carbonate (5)

¹H NMR (CDCl₃): δ 0.94 (d, 6H, J=6.4 Hz), 1.05 (d, 3H, J=6.0 Hz), 1.21 (d, 3H, J=7.2 Hz), 1.26 (m, 2H), 1.45 (s, 3H), 1.50 (m, 1H), 1.57 (m, 1H), 1.64 (m, 1H), 1.95 (m, 1H), 2.00 (m, 1H), 2.08 (m, 1H), 2.25 (m, 1H), 2.44 (m, 1H), 3.37 (m, 1H), 3.91 (dd, 2H, J=1.6, 6.4 Hz), 4.31 (dt, 1H, J=4.4, 10.8 Hz), 5.91 (s, 1H); ¹³C NMR (CDCl₃): δ 12.54, 15.36, 18.87, 24.72, 25.14, 27.76, 28.42, 32.52, 35.65, 41.40, 41.81, 47.76, 74.30, 78.33, 78.53, 93.16, 105.51, 154.92, 171.26; ESI-MS m/z [M+H]⁺C₂₀H₃₁O₈Na: 399.2.

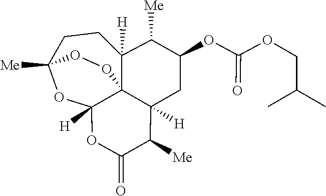 [SMILES: CC(C)COC(=O)O[C@H]1C[C@H]2[C@@H](C)C(=O)O[C@@H]3O[C@@]4(C)CC[C@@H]([C@@H]1C)[C@]32OO4]

### 3-Chloropropyl-3,6,9-trimethyl-10-oxodecahydro-3,12-epoxy[1,2]dioxepino[4,3-i]isochromen-7-yl-carbonate (7)

¹H NMR (CDCl₃): δ 1.04 (d, 3H, J=5.6 Hz), 1.21 (d, 3H, J=7.2 Hz), 1.26 (m, 1H), 1.44 (s, 3H), 1.54 (m, 1H), 1.59 (m, 1H), 1.67 (m, 1H), 1.96 (m, 1H), 1.99 (m, 1H), 2.04 (m, 1H), 2.11 (t, 2H, J=6.4 Hz), 2.25 (m, 1H), 2.44 (m, 1H), 3.38 (m, 1H), 3.62 (t, 2H, J=6.4 Hz), 4.30 (t, 2H, J=6.0 Hz), 4.47 (m, 1H), 5.90 (s, 1H). ¹³C NMR (CDCl₃): δ 12.51, 15.31, 24.67, 25.11, 28.34, 31.42, 32.48, 35.63, 40.85, 41.31, 41.74, 47.71, 68.81, 78.29, 78.91, 93.11, 105.46, 154.56, 171.61; ESI-MS m/z [M+H]⁺C₁₉H₂₈O₈: 419.1.

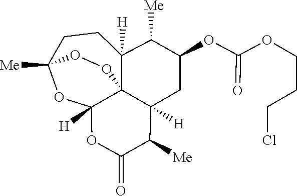 [SMILES: C[C@@H]1[C@@H](OC(=O)OCCCCl)C[C@H]2[C@@H](C)C(=O)O[C@@H]3O[C@@]4(C)CC[C@@H]1[C@]32OO4]

### Prop-2-yn-1-yl-3,6,9-trimethyl-10-oxodecahydro-3,12-epoxy[1,2]dioxepino[4,3-i]isochromen-7-yl-carbonate (8)

¹H NMR (CDCl₃): δ1.04 (d, 3H, J=6.0 Hz), 1.20 (d, 3H, J=7.2 Hz), 1.27 (m, 1H), 1.44 (s, 3H), 1.50 (m, 1H), 1.55 (m, 1H), 1.68 (m, 1H), 1.94 (m, 1H), 2.02 (m, 1H), 2.08 (m, 1H), 2.25 (m, 1H), 2.46 (m, 1H), 2.53 (s, 1H), 3.37 (m, 1H), 4.34 (dt, 1H, J=4.4, 10.8 Hz), 4.72 (s, 2H), 5.90 (s, 1H); ¹³C NMR (CDCl₃): δ 12.53, 15.29, 24.67, 25.12, 28.31, 32.49, 35.65, 41.28, 41.78, 47.74, 55.46, 75.96, 76.74, 78.27, 79.55, 93.10, 105.49, 154.12, 177.14; ESI-MS m/z [M+Na]⁺C₁₉H₂₄O₈Na: 403.1.

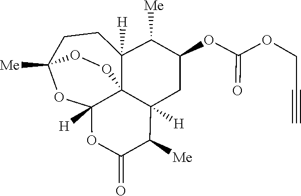 [SMILES: C#CCOC(=O)O[C@H]1C[C@H]2[C@@H](C)C(=O)O[C@@H]3O[C@@]4(C)CC[C@@H]([C@@H]1C)[C@]32OO4]

### Phenyl-3,6,9-trimethyl-10-oxodecahydro-3,12-epoxy[1,2]dioxepino[4,3-i]isochromen-7-yl-carbonate (9)

¹H NMR (CDCl₃): δ 1.12 (d, H3, J=6.0 Hz), 1.22 (d, 3H, J=7.2 Hz), 1.32 (m, 1H), 1.46 (s, 3H), 1.50 (m, 1H), 1.61 (m, 1H), 1.70 (m, 1H), 1.97 (m, 1H), 2.00 (m, 1H), 2.08 (m, 1H), 2.33 (m, 1H), 2.46 (m, 1H), 3.38 (m, 1H), 4.41 (dt, 1H, J=4.4, 10.8 Hz), 5.90 (s, 1H), 7.16 (d, 2H, J=8.0 Hz), 7.25 (t, 1H, J=7.2 Hz), 7.39 (t, 2H, J=7.6 Hz). ¹³C NMR (CDCl₃): δ 12.56, 15.38, 24.69, 25.15, 28.29, 32.52, 35.67, 41.34, 41.81, 47.74, 78.30, 79.77, 93.14, 105.52, 120.92, 126.21, 129.56, 150.93, 153.27, 171.18; ESI-MS m/z [M+Na]⁺C₂₂H₂₆O₈Na: 441.2.

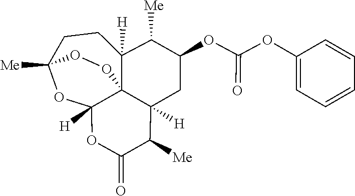 [SMILES: C[C@@H]1[C@@H](OC(=O)Oc2ccccc2)C[C@H]2[C@@H](C)C(=O)O[C@@H]3O[C@@]4(C)CC[C@@H]1[C@]32OO4]

### 4-Methylphenyl-3,6,9-trimethyl-10-oxodecahydro-3,12-epoxy[1,2]dioxepino[4,3-i]isochromen-7-yl-carbonate (10)

¹H NMR (CDCl₃): δ 1.11 (d, 3H, J=6.0 Hz), 1.22 (d, 3H, J=7.2 Hz), 1.31 (m, 1H), 1.45 (s, 3H), 1.50 (m, 1H), 1.61 (m, 1H), 1.69 (m, 1H), 1.97 (m, 1H), 2.04 (m, 1H), 2.16 (m, 1H), 2.32 (m, 1H), 2.34 (s, 3H), 2.47 (m, 1H), 3.39 (m, 1H), 4.40 (dt, 1H, J=4.4, 10.2 Hz), 5.94 (s, 1H), 7.04 (d, 2H, J=8.4 Hz), 7.17 (d, 2H, J=8.0 Hz); ¹³C NMR (CDCl₃): δ 12.56, 15.38, 20.84, 24.70, 25.15, 28.31, 32.53, 35.68, 41.36, 41.82, 47.75, 78.30, 79.66, 93.15, 105.52, 120.60, 130.02, 135.90, 148.78, 153.46, 171.18; ESI-MS m/z [M+H]⁺C₂₃H₂₉O₈: 433.2.

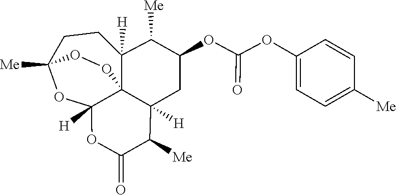 [SMILES: Cc1ccc(OC(=O)O[C@H]2C[C@H]3[C@@H](C)C(=O)O[C@@H]4O[C@@]5(C)CC[C@@H]([C@@H]2C)[C@]43OO5)cc1]

### 4-Chlorophenyl-3,6,9-trimethyl-10-oxodecahydro-3,12-epoxy[1,2]dioxepino[4,3-i]isochromen-7-yl-carbonate (11)

¹H NMR (CDCl₃): δ 1.11 (d, 3H, J=6.0 Hz), 1.22 (d, 3H, J=7.2 Hz), 1.31 (m, 1H), 1.46 (s, 3H), 1.50 (m, 1H), 1.60 (m, 1H), 1.70 (m, 1H), 1.97 (m, 1H), 2.03 (m, 1H), 2.09 (m, 1H), 2.34 (m, 1H), 2.48 (m, 1H), 3.40 (m, 1H), 4.39 (dt, 1H, J=6.0, 10.4 Hz), 5.94 (s, 1H), 7.12 (d, 2H, J=8.8 Hz), 7.35 (d, 2H, J=8.8 Hz); ¹³C NMR (CDCl₃): δ 12.55, 15.39, 24.70, 25.14, 28.30, 32.52, 35.66, 41.35, 41.83, 47.74, 78.23, 80.05, 93.14, 105.56, 122.25, 122.32, 129.61, 129.69, 131.61, 149.38, 153.00, 171.23; ESI-MS m/z [M+Na]⁺C₂₃H₂₈O₈Cl: 467.1.

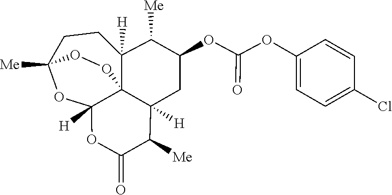 [SMILES: C[C@@H]1[C@@H](OC(=O)Oc2ccc(Cl)cc2)C[C@H]2[C@@H](C)C(=O)O[C@@H]3O[C@@]4(C)CC[C@@H]1[C@]32OO4]

### 2-Chlorophenyl-3,6,9-trimethyl-10-oxodecahydro-3,12-epoxy[1,2]dioxepino[4,3-i]isochromen-7-yl-carbonate (12)

¹H NMR (CDCl₃): δ 1.04 (d, 3H, J=6.0 Hz), 1.20 (d, 3H, J=7.2 Hz), 1.30 (m, 1H), 1.44 (s, 3H), 1.51 (m, 1H), 1.55 (m, 1H), 1.65 (m, 1H), 1.96 (m, 1H), 2.01 (m, 1H), 2.09 (m, 1H), 2.26 (m, 1H), 2.44 (m, 1H), 3.37 (m, 1H), 4.35 (dt, 1H, J=4.4, 10.8 Hz), 5.27 (d, 2H, J=2.4 Hz), 5.90 (s, 1H), 7.28 (m, 2H), 7.40 (m, 2H); ¹³C NMR (CDCl₃): δ 12.62, 15.42, 24.77, 25.21, 28.45, 32.58, 35.72, 41.45, 41.87, 47.81, 67.12, 78.36, 79.21, 93.21, 105.59, 127.08, 129.73, 129.87, 130.21, 132.77, 133.70, 154.58, 171.30; ESI-MS m/z [M+Na]⁺C₂₃H₂₈O₈Cl: 467.1.

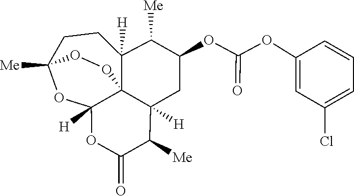 [SMILES: C[C@@H]1[C@@H](OC(=O)Oc2cccc(Cl)c2)C[C@H]2[C@@H](C)C(=O)O[C@@H]3O[C@@]4(C)CC[C@@H]1[C@]32OO4]

### 1,3-Benzodioxol-5-ylmethyl-(3R,5aS,6S,7S,8aS,9R,12S,12aR)-3,6,9-trimethyl-10-oxodecahydro-3,12-epoxy[1,2]dioxepino[4,3-i]isochromen-7-yl-carbonate (18)

¹H NMR (CDCl₃): δ 1.05 (d, 3H, J=6.0 Hz), 1.23 (d, 3H, J=7.5 Hz), 1.26 (m, 1H), 1.46 (s, 3H), 1.49 (m, 1H), 1.57 (m, 1H), 1.59-1.67 (m, 1H), 1.95-2.03 (m, 2H), 2.08-2.12 (dt, 1H, J=3.5, 7.0, 14.5 Hz), 2.25-2.29 (dt, 1H, J=4.5, 8.5, 13.0 Hz), 2.42-2.49 (ddd, 1H, J=4.0, 11.0, 13.0 Hz), 3.36-3.42 (m, 1H), 4.35 (dt, 1H, J=4.5, 11.0, 11.0 Hz), 5.07 (s, 2H), 5.92 (s, 1H) 5.98 (s, 2H), 6.80 (d, 1H, J=7.5 Hz), 6.87 (d, 2H, J=7.5 Hz); 13CNMR (CDCl₃): δ 12.96, 15.76, 25.07, 25.50, 28.77, 32.85, 35.99, 41.72, 42.15, 48.07, 70.07, 78.43, 79.07, 93.20, 101.27, 105.51, 108.29, 109.04, 122.44, 128.52, 147.71, 147.82, 154.45, 170.88; ESI-MS m/z [M+Na]⁺C₂₄H₂₈NO₁₀Na: 499.2.

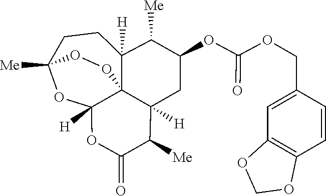 [SMILES: C[C@@H]1[C@@H](OC(=O)OCc2ccc3c(c2)OCO3)C[C@H]2[C@@H](C)C(=O)O[C@@H]3O[C@@]4(C)CC[C@@H]1[C@]32OO4]

A representative procedure for the preparation of carbamates was to a stirred solution of 7β-hydroxy artemisinin (1.0 equiv) and 4-dimethylaminopyridine (catalytic amount, typically one crystal) in dry toluene (0.25 M) was added the appropriate isocyanate (1.5 equiv), and the resulting mixture was stirred at 90° C. for 12-24 h. Reaction mixtures were concentrated under vacuum, and the crude residue was purified by flash column chromatography using ethyl acetate-hexanes. Yields of the products ranged from 50-75%.

This procedure is further shown in the diagram below:

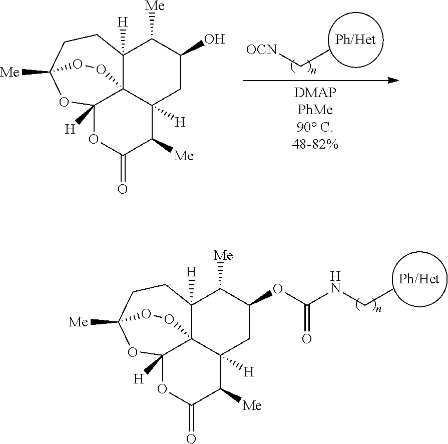

Another representative procedure used to prepare carbamates was to a flame-dried 25 mL round-bottom flask with stir bar under a dry nitrogen atmosphere, added a solution of PC-009 (1 equiv) in dry dichloromethane (0.25 M) the solution was stirred at room temperature for 10-15 minutes. The reaction was then cooled at 0° C. for an additional 15 minutes, and then methyl triflate (1.0 equiv) was cautiously added dropwise (CAUTION: methyl triflate is acutely toxic, thus extreme care must be taken when handling). After approximately 15 minutes, the reaction mixture was warmed to room temperature and allowed to react for an additional hour. The crude mixture was concentrated under vacuum to dryness (the receiving flask was filled with 2 M NaOH in an effort to quench residual MeOTf), and the crude solid was further dried overnight via a high vacuum pump to give a fine tan solid, PC-009-Methyl-Imidazolium-Triflate (>95%), which was used for the subsequent reactions without further purification. Note: The crude solid was kept under an atmosphere of nitrogen and placed in a freezer (−22° C.) to limit decomposition of the product.

To a 1 dram vial with stir bar, quickly added PC-009-Methyl-Imidazolium-Triflate (1.0 equiv) followed by dry DMSO (0.5 M), the appropriate amine/aniline (1.0 equiv), and N-Methyl-Imidazole (1.0 equiv). The reaction was allowed to stir for 15 to 30 minutes and then analyzed by TLC to ensure completion of the reaction. The reaction was then diluted with a generous portion of EtOAc transferred to a separatory funnel and a 5% LiCl (aq) solution was added. The organic phase was then washed five times with the 5% LiCl (aq) solution. The organic layer was dried with sodium sulfate and concentrated under vacuum and the crude residue was purified by flash column chromatography using ethyl acetate-hexanes.

This procedure if further shown by the diagram below:

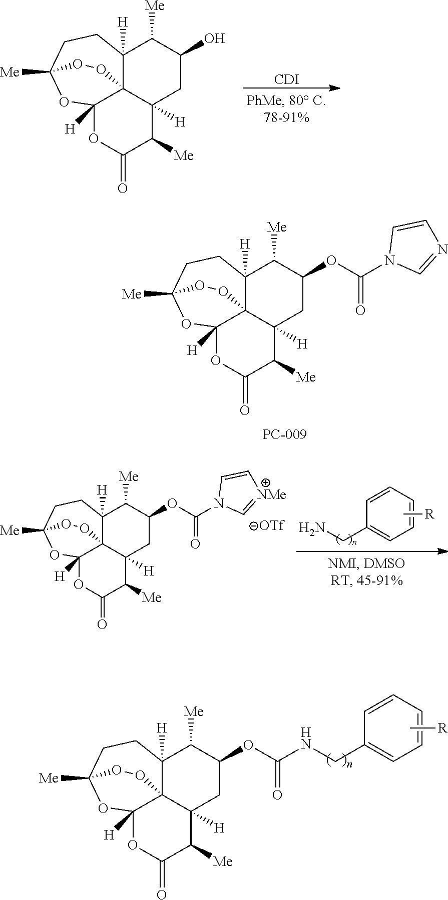

Embodiments of C-7 functionalized artemisinin derivative carbamates prepared are described and shown and described in paragraphs [0045]-[0098].

### (3R,5aS,6S,7S,8aS,9R,12S,12aR)-3,6,9-Trimethyl-10-oxodecahydro-3,12-epoxy[1,2]dioxepino[4,3-i]isochromen-7-yl-(2-piperidin-1-ylethyl) carbamate (33)

¹H NMR (CDCl₃/MeOD): δ 1.01 (d, 3H, J=3.6 Hz), 1.18 (d, 3H, J=7.2 Hz), 1.22 (m, 1H), 1.42 (s, 3H), 1.45 (m, 1H), 1.50 (m, 2H), 1.58 (m, 6H), 1.97 (m, 1H), 1.99 (m, 1H), 2.06 (m, 1H), 2.18 (dt, 1H, J=3.6, 8.4, 8.4 Hz), 2.40 (m, 1H), 2.47 (m, 6H), 3.26 (t, 2H, J=6.4), 3.32 (m, 1H), 4.31 (m, 1H), 5.96 (s, 1H); ¹³C NMR (CDCl₃/MeOD): δ 12.26, 15.11, 23.84, 24.69, 24.88, 25.25, 28.80, 32.58, 35.61, 37.35, 41.64, 41.75, 54.32, 57.96, 75.26, 78.55, 93.58, 105.52, 156.57, 172.33; □ESI-MS m/z [M+H]⁺C₂₃H₃₆NO₈:454.2.

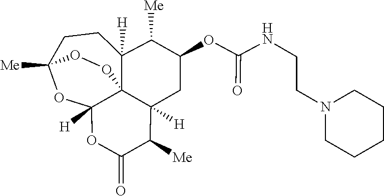 [SMILES: C[C@@H]1[C@@H](OC(=O)NCCN2CCCCC2)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-(2-pyrrolidin-1-ylethyl) carbamate (34)

¹H NMR (CDCl₃): δ1.01 (d, 3H, J=4.5 Hz), 1.19 (d, 3H, J=7.5 Hz), 1.25 (m, 1H), 1.45 (s, 3H), 1.50-1.52 (m, 1H), 1.56 (m, 2H), 1.86 (m, 4H), 1.95-2.02 (m, 2H), 2.06-2.12 (m, 1H), 2.20.2.23 (m, 1H), 2.42-2.47 (m, 1H), 2.71 (m, 4H), 2.74 (t, 2H, J=6.0), 3.35-3.38 (m, 3H), 4.36 (m, 1H), 5.75 (brs, 1H), 5.93 (s, 1H); ¹³C NMR (CDCl₃): δ 12.95, 15.80, 23.77, 25.15, 25.52, 29.32, 32.92, 36.03, 39.35, 42.05, 42.22, 48.18, 54.16, 55.30, 75.32, 78.63, 93.36, 105.46, 155.91, 171.22; ESI-MS m/z [M+H]⁺C₂₂H₃₄NO₈:440.2.

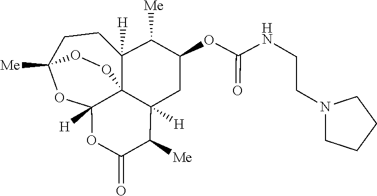 [SMILES: C[C@@H]1[C@@H](OC(=O)NCCN2CCCC2)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl(2-morpholin-4-ylethyl) carbamate (35)

¹H NMR (CDCl₃): δ 0.99 (d, 3H, J=4.0 Hz), 1.12 (m, 1H), 1.17 (d, 3H, J=7.2 Hz), 1.41 (s, 3H), 1.53 (m, 3H), 1.93 (m, 1H), 1.99 (m, 1H), 2.06 (m, 1H), 2.19 (m, 1H), 2.38 (m, 1H), 2.44 (m, 6H), 3.29 (m, 2H), 3.34 (m, 1H), 3.67 (m, 4H), 4.34 (m, 1H), 5.27 (brs, 1H), 5.89 (s, 1H); ¹³C NMR (CDCl₃): δ 12.48, 15.34, 24.78, 25.11, 28.97, 32.56, 35.71, 37.20, 41.79, 41.93, 47.90, 53.32, 57.38, 66.76, 75.11, 78.47, 93.27, 105.42, 155.92, 171.38; ESI-MS m/z [M+H]⁺C₂₂H₃₃NO₉:456.2.

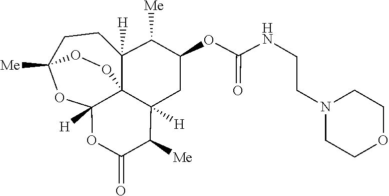 [SMILES: C[C@@H]1[C@@H](OC(=O)NCCN2CCOCC2)C[C@H]2[C@@H](C)C(=O)O[C@@H]3O[C@@]4(C)CC[C@@H]1[C@]32OO4]

### 3,6,9-Trimethyl-10-oxodecahydro-3,12-epoxy[1,2]dioxepino[4,3-i]isochromen-7-yl-phenylcarbamate (19)

¹H NMR (CDCl₃): δ 1.05 (d, 3H, J=5.1 Hz), 1.19 (d, 3H, J=7.5 Hz), 1.25 (m, 1H), 1.45 (s, 3H), 1.55 (m, 4H), 2.03 (m, 3H), 2.27 (dt, 1H, J=3.0, 8.4, 12.6 Hz), 2.46 (m 1H), 3.38 (m, 1H), 4.49 (m, 1H), 5.93 (s, 1H), 6.60 (brs, 1H), 7.07 (dd, 1H, J=7.2, 7.2 Hz), 7.33 (m, 4H); 13CNMR (CDCl₃): δ 12.51, 15.38, 24.80, 25.14, 28.93, 32.57, 35.70, 41.76, 41.96, 47.90, 75.63, 78.39, 93.22, 105.50, 118.70, 123.75, 129.10, 137.47, 152.88, 171.36; ESI-MS m/z [M+Na]⁺C₂₅H₂₈NO₁₀Na: 440.2.

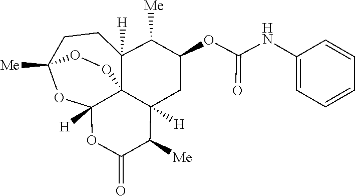 [SMILES: C[C@@H]1[C@@H](OC(=O)Nc2ccccc2)C[C@H]2[C@@H](C)C(=O)O[C@@H]3O[C@@]4(C)CC[C@@H]1[C@]32OO4]

### 3,6,9-Trimethyl-10-oxodecahydro-3,12-epoxy[1,2]dioxepino[4,3-i]isochromen-7-yl-benzylcarbamate (20)

¹H NMR (CDCl₃): δ 1.03 (d, 3H, J=5.0 Hz), 1.16 (m, 1H), 1.22 (d, 3H, J=6.5 Hz), 1.46 (s, 3H), 1.52-1.56 (m, 3H), 1.98 (m, 2H), 2.10 (m, 1H), 2.24 (m, 1H), 2.45 (m, 1H), 3.38 (m, 1H), 4.42 (m, 3H), 5.11 (brs, 1H), 5.91 (s, 1H), 7.29 (m, 3H), 7.36 (m, 2H); ¹³C NMR (CDCl₃): δ12.95, 15.76, 25.15, 25.51, 29.34, 32.92, 36.02, 42.14, 42.22, 45.37, 48.14, 75.49, 78.59, 93.33, 105.49, 127.36, 127.52, 128.63, 138.07, 155.77, 171.21; ESI-MS m/z [M+H]⁺C₂₃H₃₀NO₇:432.2.

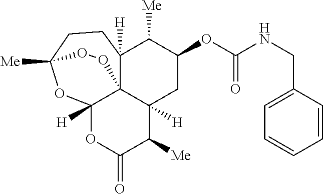 [SMILES: C[C@@H]1[C@@H](OC(=O)NCc2ccccc2)C[C@H]2[C@@H](C)C(=O)O[C@@H]3O[C@@]4(C)CC[C@@H]1[C@]32OO4]

### 3,6,9-Trimethyl-10-oxodecahydro-3,12-epoxy[1,2]dioxepino[4,3-i]isochromen-7-yl-[4-methoxy phenyl]carbamate (21)

¹H NMR (CDCl₃): δ 0.90 (d, 3H, J=5.5 Hz), 1.03 (d, 3H, J=7.5 Hz), 1.24 (m, 1H), 1.29 (s, 3H), 1.36 (m, 1H), 1.44, (m, 1H), 1.52 (m, 1H), 1.82 (m, 1H), 1.86 (m, 1H), 1.94 (ddd, 1H, J=4.0, 6.0, 14.5 Hz), 2.10 (ddd, 1H, J=4.0, 8.5, 13.0 Hz), 2.30 (dt, 1H, J=3.5, 14.0, 14.0 Hz), 3.20 (m, 1H), 3.61 (s, 3H), 4.29 (m, 1H), 5.79 (s, 1H), 6.67 (d, 2H, J=9.0 Hz), 7.18 (d, 2H, J=9.0 Hz); ¹³C NMR (CDCl₃): δ12.52, 15.39, 24.79, 25.14, 29.68, 32.57, 35.71, 41.77, 41.96, 47.90, 55.48, 75.52, 78.40, 93.23, 105.49, 114.30, 120.67, 130.55, 153.20, 156.22, 171.38; □ESI-MS m/z [M+Na]⁺C₂₃H₃₀NO₈: 470.2.

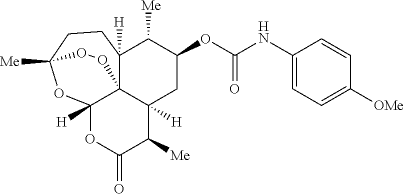 [SMILES: COc1ccc(NC(=O)O[C@H]2C[C@H]3[C@@H](C)C(=O)O[C@@H]4O[C@@]5(C)CC[C@@H]([C@@H]2C)[C@]43OO5)cc1]

### 3,6,9-Trimethyl-10-oxodecahydro-3,12-epoxy[1,2]dioxepino[4,3-i]isochromen-7-yl-[4-fluorophenyl]carbamate (22)

¹H NMR (CDCl₃): δ1.08 (d, 3H, J=5.5 Hz), 1.22 (d, 3H, J=7.5 Hz), 1.32 (m, 1H), 1.48 (s, 3H), 1.55 (m, 1H), 1.60, (m, 2H), 2.00 (dt, 1H, J=4.5, 9.5, 14.5 Hz), 2.07 (m, 1H), 2.13 (m, 1H), 2.30 (dt, 1H, J=4.0, 8.5, 13.0 Hz), 2.48 (ddd, 1H, J=4.0, 14.5, 14.5 Hz), 3.41 (m, 1H), 4.51 (dt, 1H, J=4.5, 10.5, 10.5 Hz), 5.96 (s, 1H), 6.76 (brs, 1H), 7.03 (t, 2H, J=8.5 Hz), 7.37 (m, 2H); ¹³C NMR (CDCl₃): δ 12.95, 15.80, 25.18, 25.52, 29.30, 32.92, 36.02, 42.08, 42.25, 48.14, 76.90, 78.52, 93.33, 105.57, 115.63, 115.81, 121.0, 133.5, 138.2, 153.0, 171.5; ESI-MS m/z [M+Na]⁺C₂₃H₃₀NO₇FNa: 458.2.

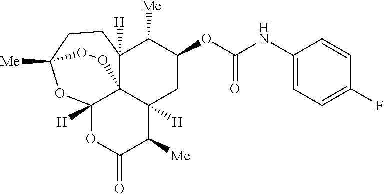 [SMILES: C[C@@H]1[C@@H](OC(=O)Nc2ccc(F)cc2)C[C@H]2[C@@H](C)C(=O)O[C@@H]3O[C@@]4(C)CC[C@@H]1[C@]32OO4]

### 3,6,9-Trimethyl-10-oxodecahydro-3,12-epoxy[1,2]dioxepino[4,3-i]isochromen-7-yl-[4-chloro phenyl]carbamate (23)

¹H NMR (CDCl₃): δ 0.96 (d, 3H, J=6.0 Hz), 1.07 (d, 3H, J=7.2 Hz), 1.14 (m, 1H), 1.33 (s, 3H), 1.41 (m, 1H), 1.49 (m, 1H), 1.55 (m, 1H), 1.90, (m, 1H), 1.94 (m, 1H), 1.98 (m, 1H), 2.15 (dt, 1H, J=4.4, 8.0, 12.8 Hz), 2.33 (ddd, 1H, J=4.0, 10.8, 13.2 Hz), 3.22 (m, 1H), 4.35 (m, 1H) 5.91 (s, 1H), 7.10 (d, 2H, J=9.2 Hz), 7.35 (d, 2H, J=8.0 Hz), 9.10 (brs, 1H); ¹³C NMR (CDCl₃): δ 12.83, 15.61, 24.88, 25.42, 29.03, 32.73, 35.87, 47.73, 47.99, 48.04, 75.56, 78.73, 93.44, 105.95, 120.23, 127.51, 128.82, 137.93, 153.68, 171.63; □ESI-MS m/z [M+H]⁺C₂₂H₂₇NO₇Cl: 452.1.

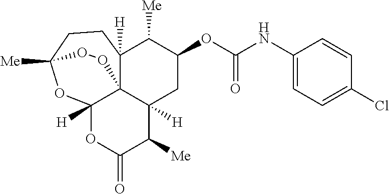 [SMILES: C[C@@H]1[C@@H](OC(=O)Nc2ccc(Cl)cc2)C[C@H]2[C@@H](C)C(=O)O[C@@H]3O[C@@]4(C)CC[C@@H]1[C@]32OO4]

### 3,6,9-Trimethyl-10-oxodecahydro-3,12-epoxy[1,2]dioxepino[4,3-i]isochromen-7-yl-[4-bromo phenyl]carbamate (24)

¹H NMR (DMSO): δ 0.93 (d, 3H, J=6.1 Hz), 1.05 (d, 3H, J=7.3 Hz), 1.26-1.34 (m, 1H), 1.35 (m, 1H), 1.37-1.46 (m, 1H), 1.58-1.77 (m, 2H), 1.88-1.95 (m, 1H), 2.02-2.13 (m, 3H), 2.26 (dd, 1H, J=8.7, 19.2 Hz), 3.10-3.18 (m, 1H), 2.49 (ddd, 1H, J=5.0, 15.0, 15.0 Hz), 4.33-4.46 (m, 1H), 6.25 (s, 1H), 7.42 (s, 4H), 9.78 (s, 1H); ¹³C NMR (DMSO): δ 12.87, 15.39, 24.59, 25.42, 28.45, 32.63, 35.81, 40.81, 41.22, 47.60, 75.78, 79.14, 93.35, 105.26, 114.43, 120.62, 131.93, 138.99, 153.57, 171.58; ESI-MS m/z [M+Na]⁺C₂₂H₂₇NO₇BrNa: 518.1.

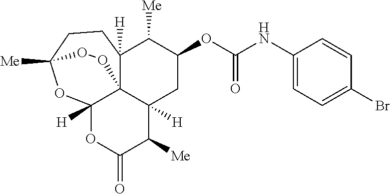 [SMILES: C[C@@H]1[C@@H](OC(=O)Nc2ccc(Br)cc2)C[C@H]2[C@@H](C)C(=O)O[C@@H]3O[C@@]4(C)CC[C@@H]1[C@]32OO4]

### 3,6,9-Trimethyl-10-oxodecahydro-3,12-epoxy[1,2]dioxepino[4,3-i]isochromen-7-yl-[3-chloro phenyl]carbamate (25)

¹H NMR (CDCl₃): δ 1.06 (d, 3H, J=5.5 Hz), 1.21 (d, 3H, J=7.0 Hz), 1.33 (m, 1H), 1.47 (s, 3H), 1.52 (m, 1H), 1.57 (m, 1H), 1.61 (m, 1H), 1.99, (m, 1H), 2.04 (m, 1H), 2.12 (m, 1H), 2.27 (dt, 1H, J=4.0, 8.5, 13.0 Hz), 2.48 (ddd, 1H, J=3.5, 13.0, 13.5 Hz), 3.41 (m, 1H), 4.51 (m, 1H) 5.94 (s, 1H), 7.01 (brs, 1H), 7.05 (m, 1H), 7.25 (m, 2H), 7.56 (m, 1H); 13C NMR (CDCl₃): δ 12.95, 15.76, 25.17, 25.51, 29.27, 32.92, 35.99, 42.08, 42.22, 48.09, 75.86, 78.50, 93.34, 105.60, 116.58, 118.64, 123.60, 129.97, 134.68, 138.77, 152.92, 171.27; DESI-MS m/z [M+H]⁺C₂₂H₂₇NO₇Cl: 452.1.

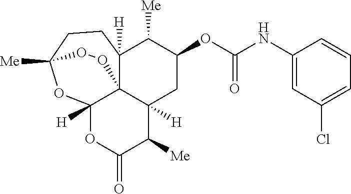 [SMILES: C[C@@H]1[C@@H](OC(=O)Nc2cccc(Cl)c2)C[C@H]2[C@@H](C)C(=O)O[C@@H]3O[C@@]4(C)CC[C@@H]1[C@]32OO4]

### 3,6,9-Trimethyl-10-oxodecahydro-3,12-epoxy[1,2]dioxepino[4,3-i]isochromen-7-yl-[2-bromo phenyl]carbamate (26)

¹H NMR (CDCl₃): δ1.09 (d, 3H, J=6.0 Hz), 1.23 (d, 3H, J=7.5 Hz), 1.31 (m, 1H), 1.47 (s, 3H), 1.53 (m, 1H), 1.66 (m, 2H), 1.96 (m, 1H), 2.03 (m, 1H), 2.10 (dt, 1H, J=3.5, 6.5, 14.5 Hz), 2.30 (ddd, 1H, J=4.5, 8.5, 13.0 Hz), 2.47 (dt, 1H, J=4.0, 14.5, 14.5 Hz), 3.40 (m, 1H), 4.52 (dt, 1H, J=4.5, 10.5, 10.5 Hz), 5.96 (s, 1H), 6.96 (dd, 1H, J=8.0 Hz), 7.15 (brs, 1H), 7.32 (dd, 1H, J=8.0 Hz), 7.53 (d, 1H, J=8.0 Hz), 8.12 (d, 1H, J=7.5 Hz); ¹³C NMR (CDCl₃): δ 12.97, 15.86, 25.15, 25.53, 29.18, 32.90, 36.03, 41.89, 42.22, 48.16, 76.26, 78.53, 93.28, 105.51, 112.2, 120.24, 124.57, 128.36, 132.28, 135.56, 152.43, 171.01; ESI-MS m/z [M+H]⁺C₂₂H₂₇NO₇Br: 496.1.

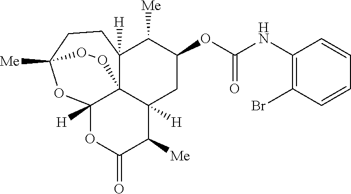 [SMILES: C[C@@H]1[C@@H](OC(=O)Nc2ccccc2Br)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-[4-(trifluoromethyl)phenyl]carbamate (27)

¹H NMR (CDCl₃): δ 1.09 (d, 3H, J=5.2 Hz), 1.18 (m, 1H), 1.23 (d, 3H, J=7.2 Hz), 1.46 (s, 3H), 1.53 (m, 1H), 1.65 (m, 2H), 2.02 (m, 2H), 2.13 (m, 1H), 2.30 (ddd, 1H, J=4.0, 9.2, 14.2 Hz), 2.49 (m, 1H), 3.42 (m, 1H), 4.54 (dt, 1H, J=3.6, 9.6, 9.6 Hz), 5.97 (s, 1H), 7.36 (brs, 1H), 7.57 (s, 4H); ¹³C NMR (CDCl₃): δ12.52, 15.32, 24.84, 25.13, 28.98, 32.63, 35.66, 41.92, 47.80, 75.80, 78.37, 93.35, 105.66, 118.22, 122.79, 125.31, 126.3, 126.35, 140.99, 152.71, 171.73; □ESI-MS m/z [M+Na]⁺C₂₃H₂₆NO₇F₃Na: 508.2.

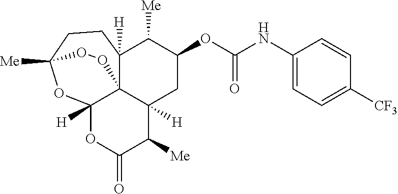 [SMILES: C[C@@H]1[C@@H](OC(=O)Nc2ccc(C(F)(F)F)cc2)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-[2-(trifluoromethyl)phenyl]carbamate (28)

¹H NMR (CDCl₃): δ1.05 (d, 3H, J=5.8 Hz), 1.21 (d, 3H, J=7.3 Hz), 1.27 (m, 2H), 1.60 (m, 4H), 2.01 (m, 1H), 2.09 (m, 1H, J=3.0), 2.27 (dt, 1H, J=4.2, 13.0 Hz), 2.45 (m, 1H), 3.38 (dd, 1H, J=5.8, 7.8 Hz), 4.49 (td, 1H, J=4.6, 10.7 Hz), 5.94 (s, 1H), 6.88 (s, 1H), 7.20 (dd, 1H, J=7.6 Hz), 7.56 (m, 2H), 8.04 (d, 1H, J=7.8 Hz); ¹³C NMR (CDCl₃): δ 12.52, 15.33, 24.76, 25.13, 28.76, 32.54, 35.69, 41.57, 41.95, 47.89, 76.33, 78.35, 93.18, 105.48, 123.97, 126.10, 126.15, 132.96, 135.19, 152.85, 171.22; ESI-MS m/z [M+H]⁺C₂₃H₂₇NO₇F₃: 486.2.

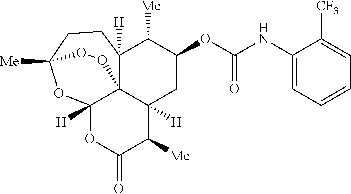 [SMILES: C[C@@H]1[C@@H](OC(=O)Nc2ccccc2C(F)(F)F)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-[2,5-difluorophenyl]carbamate (29)

¹H NMR (CDCl₃): δ1.07 (d, 3H, J=5.6 Hz), 1.22 (d, 3H, J=7.3 Hz), 1.27 (m, 1H), 1.49 (m, 4H), 1.63 (m, 2H), 1.98 (m, 2H), 2.01 (m, 1H), 2.27 (dt, 1H, 4.3, 13.0 Hz), 2.48 (m, 1H), 3.46 (m, 1H), 4.50 dd, 1H, J=6.1, 10.5 Hz), 5.93 (s, 1H), 6.53 (ddd, 1H, J=2.2, 5.5, 8.9 Hz), 6.82 (s, 1H), 7.00 (d, 2H, J=6.9 Hz); ¹³C NMR (CDCl₃): δ12.49, 15.35, 24.78, 25.12, 28.86, 32.54, 35.66, 41.70, 41.93, 47.83, 76.14, 78.27, 93.07, 105.29, 115.34, 127.25, 139.86, 152.29, 162.15, 164.60, 171.30; □ ESI-MS m/z [M+Na]⁺C₂₂H₂₅NF₂O₇:476.1.

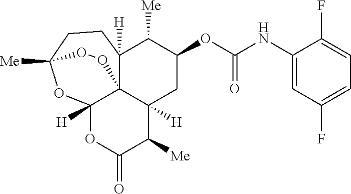 [SMILES: C[C@@H]1[C@@H](OC(=O)Nc2cc(F)ccc2F)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-[3,5-difluorophenyl]carbamate (30)

¹H NMR (CDCl₃): δ1.08 (d, 3H, J=5.6 Hz), 1.16 (m, 1H), 1.23 (d, 3H, J=7.2 Hz), 1.46 (s, 3H), 1.53 (m, 1H), 1.62 (m, 2H), 2.03 (m, 2H), 2.13 (m, 1H), 2.28 (ddd, 1H, J=4.0, 8.4, 12.8 Hz), 2.48 (ddd, 1H, J=3.6, 14.8, 14.8 Hz), 3.41 (m, 1H), 4.53 (dt, 1H, J=4.4, 10.8, 10.8 Hz), 5.96 (s, 1H), 6.51 (m, 1H), 7.05 (d, 2H, 7.2 Hz), 7.40 (brs, 1H); ¹³C NMR (CDCl₃): δ 12.47, 15.26, 24.84, 25.10, 28.93, 32.63, 35.68, 41.89, 41.94, 47.82, 75.89, 78.36, 93.35, 98.65, 101.53, 101.83, 105.64, 140.27, 152.56, 162.16, 164.61, 171.70; ESI-MS m/z [M+H]⁺C₂₂H₂₆NF₂O₇: 454.2.

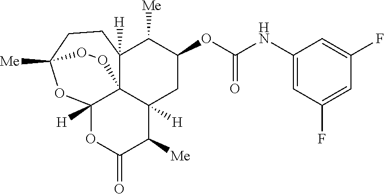 [SMILES: C[C@@H]1[C@@H](OC(=O)Nc2cc(F)cc(F)c2)C[C@H]2[C@@H](C)C(=O)O[C@@H]3O[C@@]4(C)CC[C@@H]1[C@]32OO4]

### 3,6,9-Trimethyl-10-oxodecahydro-3,12-epoxy[1,2]dioxepino[4,3-i]isochromen-7-yl-[4-chloro-2-(trifluoromethyl)phenyl]carbamate (31)

¹H NMR (CDCl₃): δ1.06 (d, 3H, J=5.6 Hz), 1.22 (d, 3H, J=7.2 Hz), 1.30 (m, 1H), 1.46 (s, 3H), 1.54, (m, 1H), 1.60 (m, 1H), 1.64 (m, 1H), 1.98 (m, 1H), 2.00 (m, 1H), 2.04 (m, 1H), 2.10, (m, 1H), 2.28 (ddd, 1H, J=3.6, 8.0, 12.8 Hz), 2.48 (ddd, 1H, 3.6, 10.8, 10.8 Hz), 3.39 (m, 1H), 4.50 (dt, 1H, J=4.4, 10.8, 10.8 Hz), 5.95 (s, 1H), 6.89 (brs, 1H), 7.53 (d, 1H, J=8.8 Hz), 7.57 (s, 1H), 8.03 (d, 1H, J=8.0 Hz); ¹³C NMR (CDCl₃): δ 12.52, 15.34, 24.76, 25.13, 28.73, 32.54, 35.69, 41.53, 41.93, 47.86, 76.64, 78.34, 93.19, 105.51, 121.76, 124.47, 126.19, 126.30, 129.47, 132.95, 133.89, 152.73, 171.24; ESI-MS m/z [M+H]⁺C₂₃H₂₆NF₃O₇:520.1.

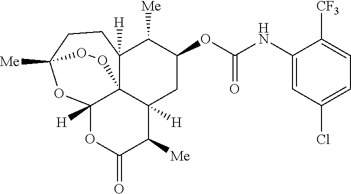 [SMILES: C[C@@H]1[C@@H](OC(=O)Nc2cc(Cl)ccc2C(F)(F)F)C[C@H]2[C@@H](C)C(=O)O[C@@H]3O[C@@]4(C)CC[C@@H]1[C@]32OO4]

### 3,6,9-Trimethyl-10-oxodecahydro-3,12-epoxy[1,2]dioxepino[4,3-i]isochromen-7-yl-[2-chloro-5-(trifluoromethyl)phenyl]carbamate (32)

¹H NMR (CDCl₃): δ 1.05 (d, 3H, J=5.8 Hz), 1.21 (d, 3H, J=7.3 Hz), 1.31 (m, 2H), 1.54 (s, 3H), 1.68 (m, 5H), 2.02 (m, 3H), 2.09, (m, 1H), 2.26 (dt, 1H, J=4.3, 12.9 Hz), 2.45 (m, 1H), 3.34-3.43 (m, 1H), 4.48 (td, 1H, J=4.6, 10.7 Hz), 5.93 (s, 1H), 6.84 (s, 1H), 7.51 (dd, 1H, J=8.9, 2.1 Hz), 7.57 (d, 1H, J=2.1 Hz), 8.04 (d, 1H, J=8.4 Hz); ¹³C NMR (CDCl₃): δ 12.56, 15.39, 24.80, 25.16, 28.78, 29.71, 32.57, 35.72, 41.57, 41.99, 47.91, 77.22, 78.34, 93.19, 105.54, 121.78, 124.50, 126.27, 126.33, 129.48, 132.98, 133.88, 152.69, 171.21; ESI-MS m/z [M+Na]⁺C₂₃H₂₅NF₃O₇Na: 542.1.

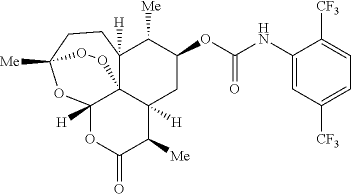 [SMILES: C[C@@H]1[C@@H](OC(=O)Nc2cc(C(F)(F)F)ccc2C(F)(F)F)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-(2-phenylethyl) carbamate (36)

¹H NMR (CDCl₃): δ 0.98 (d, 3H, J=5.7 Hz), 1.19 (d, 3H, J=7.3 Hz) 1.24 (m, 3H), 1.43 (s, 3H), 1.53 (m, 3H), 2.16 (m, 3H), 2.42 (m, 1H), 2.80 (t, 2H, J=6.8 Hz), 3.38 (m, 3H), 3.44 (m, 2H), 4.35 (td, 1H, J=4.5, 10.7 Hz), 4.67 (brs, 1H), 5.88 (s, 1H), 7.16 (d, 2H, J=7.3 Hz), 7.25 (m, 3H); ¹³C NMR (CDCl₃): δ 12.50, 15.30, 24.78, 25.13, 28.95, 29.66, 32.57, 35.70, 36.02, 41.92, 42.09, 47.87, 75.07, 78.44, 93.24, 105.44, 126.55, 128.62, 128.74, 138.52, 155.80, 171.14; ESI-MS m/z [M+Na]⁺C₂₄H₃₁NO₇Na: 468.2.

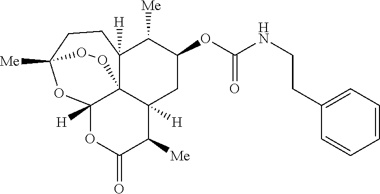 [SMILES: C[C@@H]1[C@@H](OC(=O)NCCc2ccccc2)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-[2-(4-methoxyphenyl)ethyl]carbamate (37)

¹H NMR (CDCl₃): δ 0.99 (d, 3H, J=6.0 Hz), 1.13 (m, 1H), 1.20 (d, 3H, J=7.5 Hz), 1.45 (s, 3H), 1.50 (m, 1H), 1.58 (m, 2H), 1.95 (dt, 1H, J=4.0, 9.5, 14.0 Hz), 2.01 (m, 1H), 2.07 (m, 1H), 2.19 (dt, 1H, J=4.5, 8.5, 13.0 Hz), 2.45 (dt, 1H, J=4.0, 15.0, 15.0 Hz), 2.76 (t, 2H, J=7.0 Hz), 3.37 (m, 3H), 3.79 (s, 3H), 4.37 (dt, 1H, J=4.5, 10.5, 10.5 Hz), 4.76 (t, 1H), 5.90 (s, 1H), 6.86 (d, 2H, J=8.5 Hz), 7.11 (d, 2H, J=8.5 Hz); ¹³C NMR (CDCl₃): δ 12.94, 15.72, 25.16, 25.51, 29.32, 32.92, 35.42, 36.03, 42.08, 42.21, 42.59, 48.14, 55.48, 75.17, 78.60, 93.34, 105.48, 114.02, 114.07, 129.60, 130.40, 155.63, 158.09, 171.21; □ESI-MS m/z [M+H]⁺C₂₅H₃₄NO₈:476.2.

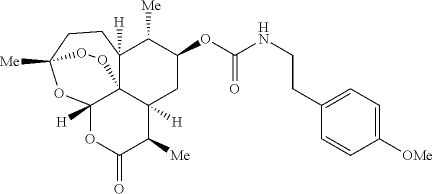 [SMILES: COc1ccc(CCNC(=O)O[C@H]2C[C@H]3[C@@H](C)C(=O)O[C@@H]4O[C@@]5(C)CC[C@@H]([C@@H]2C)[C@]43OO5)cc1]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-(cyclopropylmethyl) carbamate (39)

¹H NMR (CDCl₃): δ 0.16 (d, 2H, J=4.0 Hz), 0.49, (q, 2H), 0.95 (m, 1H), 1.00 (d, 3H, J=5.2 Hz), 1.12 (m, 1H), 1.17 (d, 3H, J=7.2 Hz), 1.42 (s, 3H), 1.53 (m, 3H), 1.93 (ddd, 1H, J=4.4, 9.2, 14.4 Hz), 2.01 (m, 2H), 2.17 (m, 1H), 2.43 (ddd, 1H, J=4.0, 12.8, 14.2 Hz), 2.07 (m, 2H), 3.32 (m, 1H), 4.35 (m, 1H), 4.94 (brs, 1H), 5.91 (s, 1H); ¹³C NMR (CDCl₃): δ 3.20, 10.93, 12.48, 15.30, 24.79, 25.12, 28.99, 32.58, 35.72, 41.88, 41.95, 45.87, 47.92, 74.98, 78.50, 93.28, 105.44, 155.86, 171.43; ESI-MS m/z [M+H]⁺C₂₀H₃₀NO₇:396.2.

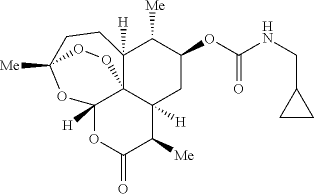 [SMILES: C[C@@H]1[C@@H](OC(=O)NCC2CC2)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-4-methylpiperazine-1-carboxylate (41)

¹H NMR (CDCl₃): δ 0.99 (d, 3H, J=5.2 Hz), 1.13 (m, 1H), 1.18 (d, 3H, J=7.2 Hz), 1.42 (s, 3H), 1.49 (m, 1H), 1.56 (m, 2H), 1.95 (dt, 1H, J=4.4, 9.2, 9.2 Hz), 2.04 (m, 1H), 2.07 (m, 1H), 2.20 (dt, 1H, J=4.0, 8.0, 8.0 Hz), 2.29 (s, 3H), 2.36 (m, 4H), 2.45 (m, 1H), 3.34 (m, 1H), 3.47 (m, 4H), 4.37 (dt, 1H, J=4.4, 10.4, 10.4 Hz), 5.92 (s, 1H); ¹³C NMR (CDCl₃): δ 12.49, 15.38, 24.79, 25.12, 29.03, 32.58, 35.72, 41.79, 41.96, 43.60, 46.01, 47.89, 54.59, 75.88, 78.46, 93.26, 105.41, 154.62, 171.35; ESI-MS m/z [M+H]⁺C₂₁H₃₃N₂O₈:425.2.

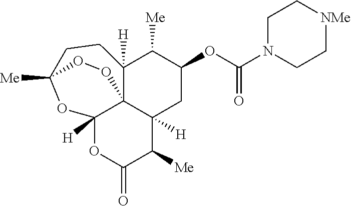 [SMILES: C[C@@H]1[C@@H](OC(=O)N2CCN(C)CC2)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-pyrrolidine-1-carboxylate (42)

¹H NMR (CDCl₃): δ 1.03 (d, 3H, J=5.6 Hz), 1.20 (d, 3H, J=7.2 Hz), 1.23 (m, 1H), 1.44 (s, 3H), 1.58 (m, 3H), 1.86 (m, 4H), 1.97 (dt, 1H, J=4.4, 9.6, 9.6 Hz), 2.08 (m, 2H), 2.27 (ddd, 1H, J=4.4, 8.8, 12.8 Hz), 2.45 (ddd, 1H, J=4.0, 10.8, 16.0 Hz), 3.36 (m, 5H), 4.38 (dt, 1H, J=4.8, 10.8, 10.8 Hz), 5.94 (s, 1H); ¹³C NMR (CDCl₃): δ 12.50, 15.41, 24.83, 25.14, 25.64, 29.25, 32.61, 35.76, 41.87, 42.01, 45.71, 46.19, 47.96, 75.30, 78.55, 93.32, 105.41, 154.37, 171.46; ESI-MS m/z [M+H]⁺C₂₀H₃₀NO₇:396.2.

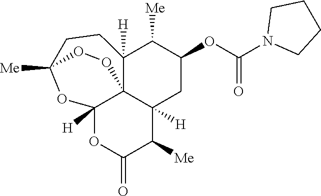 [SMILES: C[C@@H]1[C@@H](OC(=O)N2CCCC2)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl-piperidine-1-carboxylate (43)

¹H NMR (CDCl₃): δ 1.00 (d, 3H, J=4.8 Hz), 1.16 (m, 1H), 1.18 (d, 3H, J=7.2 Hz), 1.43 (s, 3H), 1.54 (m, 3H), 1.58 (m, 6H), 2.02 (m, 3H), 2.23 (dt, 1H, J=3.6, 7.6, 7.6 Hz), 2.44 (m, 1H), 3.38 (m, 5H), 4.38 (dt, 1H, J=4.4, 10.4, 10.4 Hz), 5.93 (s, 1H); ¹³C NMR (CDCl₃): δ 12.50, 15.37, 24.30, 24.82, 25.13, 25.65, 29.10, 32.61, 35.75, 41.86, 42.00, 44.84, 47.94, 75.54, 78.53, 93.30, 105.41, 154.75, 171.43; HRMS (ESI) m/z: calculated for C₂₁H₃₂NO₇ [M+H]⁺410.2179, found 410.2162 [M+H]⁺. ESI-MS m/z [M+H]⁺C₂₁H₃₂NO₇:410.2.

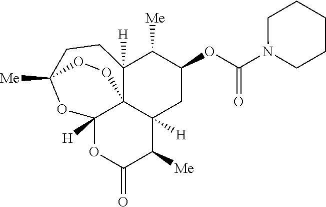 [SMILES: C[C@@H]1[C@@H](OC(=O)N2CCCCC2)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-Trimethyl-10-oxodecahydro-3,12-epoxy[1,2]dioxepino[4,3-i]isochromen-7-yl 1H-imidazole-1-carboxylate (44)

¹H NMR (CDCl₃): δ 1.10 (d, 3H, J=6.3 Hz), 1.23 (d, 3H, J=7.3 Hz), 1.33-1.45 (m, 2H), 1.48 (s, 3H), 1.58-1.71 (m, 2H), 1.82 (d, 1H, J=6.2 Hz), 2.01-2.09 (m, 2H), 2.13 (d, 1H, J=15.5 Hz), 2.32-2.41 (m, 1H), 3.39-3.47 (m, 1H), 4.71 (ddd, 1H, J=4.0, 10.8, 14.8 Hz), 5.98 (s, 1H), 7.10 (s, 1H), 7.40, (s, 1H), 8.12 (s, 1H); ¹³CNMR (CDCl₃): δ 12.50, 15.40, 24.67, 25.09, 28.40, 32.46, 35.62, 41.28, 41.87, 47.79, 78.07, 79.62, 93.00, 105.54, 116.98, 130.85, 136.95, 148.19, 170.85; HRMS (ESI) m/z: calculated for C₁₉H₂₅N₂O₇ [M+H]⁺393.1662, found 393.1669 [M+H]⁺. ESI-MS m/z [M+H]⁺C₁₉H₂₅N₂O₇:393.2.

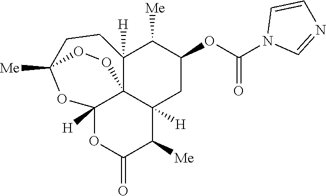 [SMILES: C[C@@H]1[C@@H](OC(=O)n2ccnc2)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-trimethyl-10-oxodecahydro-12H-3,12-epoxy[1,2]dioxepino[4,3-i]isochromen-7-yl (4-chloro-2-fluorophenyl) carbamate (PC-029)

¹H NMR (400 MHZ, CDCl₃) δ8.01 (s, 1H), 7.19-7.07 (m, 2H), 6.78 (s, 1H), 5.94 (d, J=2.3 Hz, 1H), 4.50 (s, 1H), 3.45-3.36 (m, 1H), 2.47 (td, J=14.0, 12.9, 3.3 Hz, 1H), 2.32-2.25 (m, 1H), 2.15-1.95 (m, 4H), 1.47 (d, J=2.4 Hz, 3H), 1.26 (s, 3H), 1.21 (d, J=2.4 Hz, 4H), 1.09-1.05 (m, 3H); ¹³C NMR (100 MHZ, CDCl₃) δ 124.84, 105.52, 93.17, 77.30, 76.98, 76.66, 47.90, 41.95, 41.62, 35.70, 32.54, 28.82, 25.14, 24.77, 15.40, 12.52. ESI-MS m/z [M+H]⁺C₂₂H₂₆ClF NO₇:470.1.

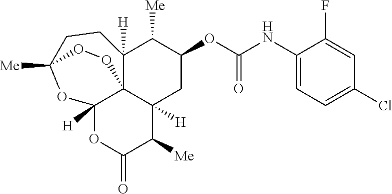 [SMILES: C[C@@H]1[C@@H](OC(=O)Nc2ccc(Cl)cc2F)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-trimethyl-10-oxodecahydro-12H-3,12-epoxy[1,2]dioxepino[4,3-i]isochromen-7-yl (2,5-difluorobenzyl) carbamate (PC-037)

¹H NMR (500 MHZ, CDCl₃) δ 7.01-6.81 (m, 3H), 5.84 (s, 1H), 5.03 (t, J=6.5 Hz, 1H), 4.41-4.20 (m, 3H), 3.31 (p, J=7.0 Hz, 1H), 2.46-2.33 (m, 1H), 2.22-2.09 (m, 1H), 2.05-1.96 (m, 1H), 1.96-1.80 (m, 2H), 1.57-1.41 (m, 5H), 1.38 (s, 3H), 1.14 (d, J=7.2 Hz, 3H), 0.95 (d, J=5.4 Hz, 3H); ¹³C NMR (100 MHz, CDCl₃) δ 171.44, 115.78, 105.52, 93.25, 75.65, 47.88, 41.96, 41.84, 38.99, 35.72, 32.59, 28.98, 25.17, 24.80, 15.35, 12.54. ESI-MS m/z [M+H]⁺C₂₃H₂₈F₂NO₇:468.2.

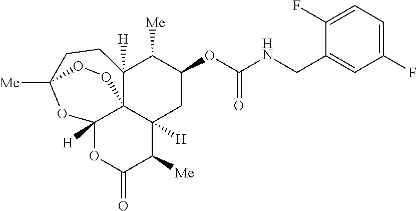 [SMILES: C[C@@H]1[C@@H](OC(=O)NCc2cc(F)ccc2F)C[C@H]2[C@@H](C)C(=O)O[C@@H]3O[C@@]4(C)CC[C@@H]1[C@]32OO4]

### (3R,5aS,6S,7S,8aS,9R,12S,12aR)-3,6,9-trimethyl-10-oxodecahydro-12H-3,12-epoxy[1,2]dioxepino[4,3-i]isochromen-7-yl (4-cyanobenzyl) carbamate (PC-40)

¹H NMR (500 MHZ, CDCl₃) δ 7.64 (d, J=7.8 Hz, 2H), 7.39 (d, J=7.9 Hz, 2H), 5.90 (s, 1H), 5.19 (t, J=6.5 Hz, 1H), 4.50-4.35 (m, 3H), 3.38 (p, J=6.7 Hz, 1H), 2.52-2.39 (m, 1H), 2.22 (dt, J=13.0, 4.3 Hz, 1H), 2.11-2.05 (m, 1H), 2.05-1.93 (m, 2H), 1.56-1.48 (m, 2H), 1.45 (s, 3H), 1.28-1.23 (m, 1H), 1.20 (d, J=7.2 Hz, 3H), 1.17-1.11 (m, 1H), 1.02 (d, J=5.4 Hz, 3H). ESI-MS m/z [M+H]⁺C₂₄H₂₉N₂O₇:456.2.

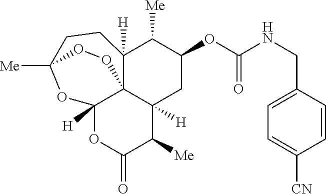 [SMILES: C[C@@H]1[C@@H](OC(=O)NCc2ccc(C#N)cc2)C[C@H]2[C@@H](C)C(=O)O[C@@H]3O[C@@]4(C)CC[C@@H]1[C@]32OO4]

A representative procedure used to prepare deoxoartemisinin carbamates was to a stirred solution of 7β-hydroxy-10-deoxoartemisinin (1.0 equiv) and 4-dimethylaminopyridine (catalytic amount, typically one crystal) in dry toluene (0.25 M) was added the appropriate isocyanate (1.5 equiv), and the resulting mixture was stirred at 90° C. for 12-24 h. Reaction mixtures were concentrated under vacuum, and the crude residue was purified by flash column chromatography using ethyl acetate-hexanes. Yields of the products ranged from 50-75%.

This procedure is further depicted by the following diagram:

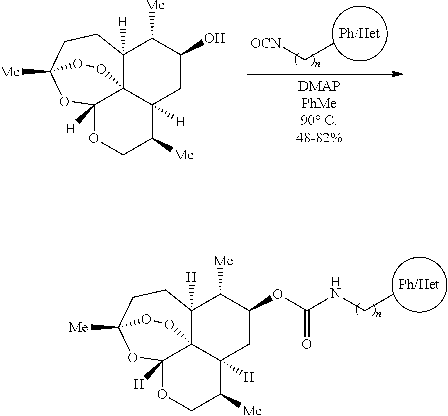

Another representative procedure used to prepare deoxoartemisinin carbamates was to a flame-dried 25 mL round-bottom flask with stir bar under a dry nitrogen atmosphere, added a solution of PC-066 (1 equiv) in dry dichloromethane (0.25 M) the solution was stirred at room temperature for 10-15 minutes. The reaction was then cooled at 0° C. for an additional 15 minutes, and then methyl triflate (1.0 equiv) was cautiously added dropwise (CAUTION: methyl triflate is acutely toxic, thus extreme care must be taken when handling). After approximately 15 minutes, the reaction mixture was warmed to room temperature and allowed to react for an additional hour. The crude mixture was concentrated under vacuum to dryness (the receiving flask was filled with 2 M NaOH in an effort to quench residual MeOTf), and the crude solid was further dried overnight via a high vacuum pump to give a fine tan solid, PC-066-Methyl-Imidazolium-Triflate (>95%), which was used for the subsequent reactions without further purification. Note: The crude solid was kept under an atmosphere of nitrogen and placed in a freezer (−22° C.) to limit decomposition of the product.

To a 1 dram vial with stir bar, quickly added PC-066-Methyl-Imidazolium-Triflate (1.0 equiv) followed by dry DMSO (0.5 M), the appropriate amine/aniline (1.0 equiv), and N-Methyl-Imidazole (1.0 equiv). The reaction was allowed to stir for 15 to 30 minutes and then analyzed by TLC to ensure completion of the reaction. The reaction was then diluted with a generous portion of EtOAc transferred to a separatory funnel and a 5% LiCl (aq) solution was added. The organic phase was then washed five times with the 5% LiCl (aq) solution. The organic layer was dried with sodium sulfate and concentrated under vacuum and the crude residue was purified by flash column chromatography using ethyl acetate-hexanes.

This procedure is further depicted by the following diagrams:

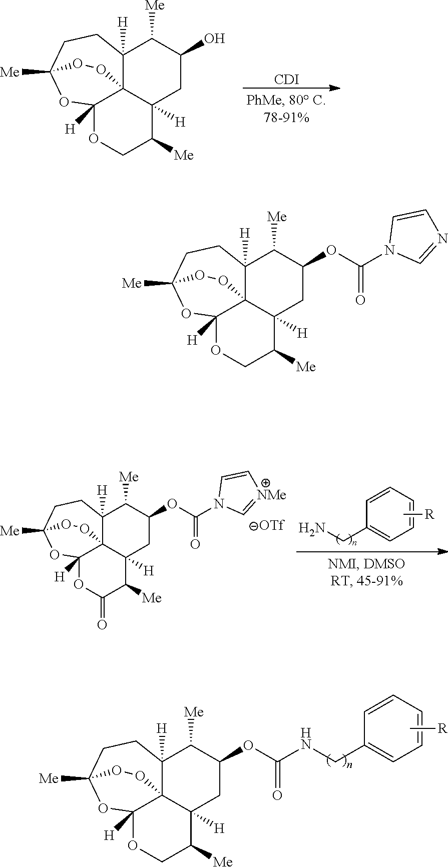

Embodiments of deoxoartemisinin carbonates produced and tested are described and shown in paragraphs [0105]-[0110].

### (3R,5aS,6S,7S,8aS,9R,12R,12aS)-3,6,9-trimethyldecahydro-12H-3,12-epoxy[1,2]dioxepino[4,3-i]isochromen-7-yl 1H-imidazole-1-carboxylate (PC-066)

¹H NMR (300 MHz, CDCl₃) δ 8.74 (s, 1H), 7.50 (s, 1H), 7.23 (s, 1H), 5.25 (s, 1H), 4.79-4.64 (m, 1H), 3.72 (dd, J=12.0, 4.2 Hz, 1H), 3.40 (t, J=11.8 Hz, 1H), 2.72-2.53 (m, 1H), 2.44-2.27 (m, 1H), 2.12-1.93 (m, 2H), 1.93-1.63 (m, 4H), 1.63-1.40 (m, 2H), 1.39 (s, 3H), 1.26-1.14 (m, 1H), 1.00 (d, J=6.2 Hz, 3H), 0.74 (d, J=7.2 Hz, 3H). ESI-MS m/z [M+H]⁺C₁₉H₂₇N₂O₆:379.2.

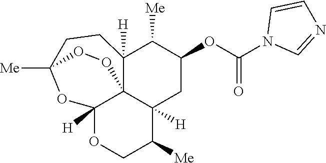 [SMILES: C[C@@H]1[C@@H](OC(=O)n2ccnc2)C[C@H]2[C@@H](C)CO[C@@H]3O[C@@]4(C)CC[C@@H]1[C@]32OO4]

### (3R,5aS,6S,7S,8aS,9R,12R,12aS)-3,6,9-trimethyldecahydro-12H-3,12-epoxy[1,2]dioxepino[4,3-i]isochromen-7-yl (3,5-difluorophenyl) carbamate (PC-076)

¹H NMR (300 MHZ, CDCl₃) δ 7.04-6.98 (m, 2H), 6.97-6.92 (m, 1H), 6.52 (ddt, J=11.2, 8.8, 2.3 Hz, 1H), 5.28 (s, 1H), 4.52 (td, J=10.5, 4.4 Hz, 1H), 3.77 (dd, J=11.7, 4.2 Hz, 1H), 3.44 (t, J=11.8 Hz, 1H), 2.75-2.61 (m, 1H), 2.49-2.35 (m, 1H), 2.33-2.13 (m, 1H), 2.11-1.99 (m, 2H), 1.96-1.88 (m, 1H), 1.81 (dt, J=14.2, 4.5 Hz, 1H), 1.64-1.58 (m, 2H), 1.59-1.48 (m, 3H), 1.45 (d, J=7.8 Hz, 3H), 1.23 (d, J=7.3 Hz, 1H), 1.03 (d, J=5.7 Hz, 3H), 0.79 (d, J=7.2 Hz, 2H). ESI-MS m/z [M+H]⁺C₂₂H₂₈NF₂O₆:440.2.

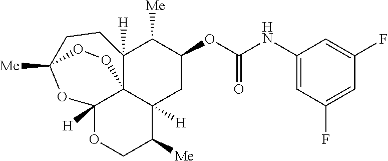 [SMILES: C[C@@H]1[C@@H](OC(=O)Nc2cc(F)cc(F)c2)C[C@H]2[C@@H](C)CO[C@@H]3O[C@@]4(C)CC[C@@H]1[C@]32OO4]

### (3R,5aS,6S,7S,8aS,9R,12R,12aS)-3,6,9-trimethyldecahydro-12H-3,12-epoxy[1,2]dioxepino[4,3-i]isochromen-7-yl (4-methylbenzyl) carbamate (PC-079)

¹H NMR (300 MHZ, CDCl₃) δ 7.21-7.09 (m, 4H), 5.22 (s, 1H), 4.96 (s, 1H), 4.42 (td, J=10.5, 4.4 Hz, 1H), 4.32 (d, J=5.7 Hz, 2H), 3.74 (dd, J=11.8, 4.1 Hz, 1H), 3.42 (t, J=11.8 Hz, 1H), 2.70-2.57 (m, 1H), 2.33 (s, 3H), 1.93-1.81 (m, 1H), 1.78 (s, 1H), 1.42 (s, 3H), 1.00-0.92 (m, 3H), 0.88 (d, J=6.4 Hz, 3H), 0.85-0.84 (m, 1H), 0.77 (d, J=7.2 Hz, 3H). ¹³C NMR (75 MHz, CDCl₃) δ 156.84, 137.40, 135.48, 129.50, 127.65, 104.40, 91.87, 79.77, 66.20, 49.78, 42.38, 41.68, 36.12, 31.71, 31.06, 27.77, 26.85, 26.15, 24.76, 22.78, 21.21, 15.50, 14.32, 14.25, 13.14. ESI-MS m/z [M+H]⁺C₂₄H₃₄NO₆:432.2.

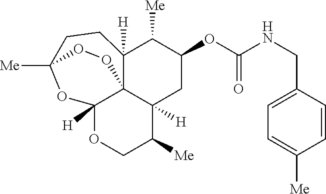 [SMILES: Cc1ccc(CNC(=O)O[C@H]2C[C@H]3[C@@H](C)CO[C@@H]4O[C@@]5(C)CC[C@@H]([C@@H]2C)[C@]43OO5)cc1]

A general procedure used for the preparation of deoxoartemisinin urea analogs is referred to as the Dess-Martin Periodinane Oxidation General Procedure. To a stirred solution of 10-Deoxo-7β-hydroxy artemisinin (II, 1.0 equiv) in dry dichloromethane (0.25 M) added Dess-Martin Periodinane (1.1 equiv) and the resulting mixture was stirred at room temperature for approximately one hour. The mixture was then diluted with 2-MeTHF and washed with DI water once and twice with a saturated solution of brine. The organic layer was collected, dried with sodium sulfate, concentrated under vacuum, and the crude residue was purified by flash column chromatography using ethyl acetate-hexanes. The desired product was isolated as a white crystal (92% yield).

This procedure is further depicted by the diagram below:

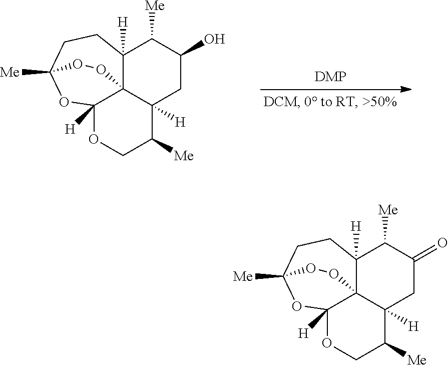 [SMILES: C[C@@H]1[C@@H](O)C[C@H]2[C@@H](C)CO[C@@H]3O[C@@]4(C)CC[C@@H]1[C@]32OO4 ; C[C@@H]1C(=O)C[C@H]2[C@@H](C)CO[C@@H]3O[C@@]4(C)CC[C@@H]1[C@]32OO4]

Another general procedure used for the preparation of deoxoartemisinin urea analogs is referred to as Reductive Amination of 10-Deoxo-Artemis-7-one. A mixture of the ketone (1.0 equiv), titanium (IV) isopropoxide (2.0 equiv) and 2.0 M ammonia in ethyl alcohol (0.2 M) was stirred under argon in a capped flask at ambient temperature for 12 hours. Sodium borohydride (1.5 equiv) was then added and the resulting mixture was stirred at room temperature for an additional 6 hours. The reaction was then concentrated down under vacuum, diluted with dichloromethane, and pre-absorbed onto silica gel. The crude residue was purified by flash column chromatography using ethyl acetate-dichloromethane for 6 CV and then the solvent system was changed to methanol-dichloromethane to give ˜50 mg of an inseparable mixture of both the α and β primary amine, which was then subjected to the same conditions indicated under the representative procedure used to prepare carbonates with 2,6 lutidine and chloroformates. The final reactional mixture was purified via column chromatography to give both α and β urea analogs.

This procedure if further depicted by the diagrams below:

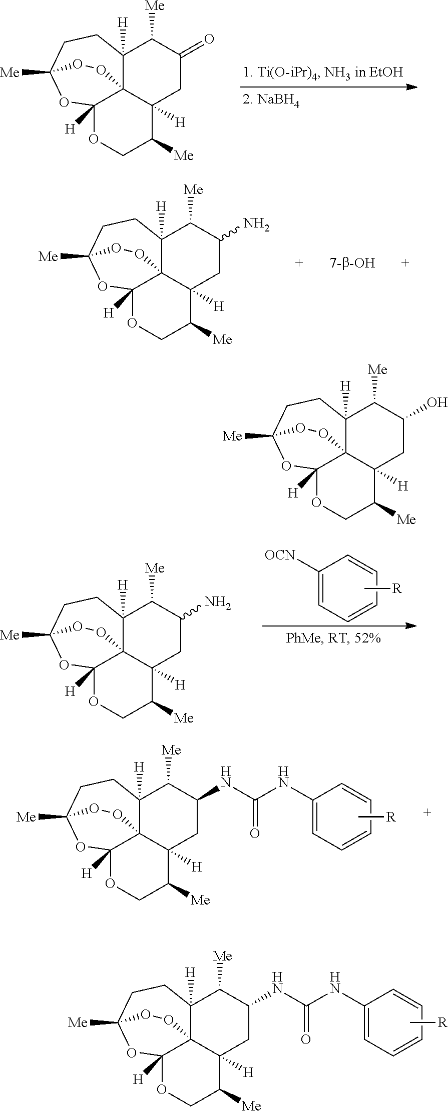

Embodiments of deoxoartemisinin urea analogs prepared and tested are described and shown in paragraphs [0116]-[0119].

### 1-(3,5-difluorophenyl)-3-((3R,5aS,6S,7R,8aS,9R,12R,12aR)-3,6,9-trimethyldecahydro-12H-3,12-epoxy[1,2]dioxepino[4,3-i]isochromen-7-yl) urea (PC-100)

¹H NMR (400 MHZ, CDCl₃) δ 7.51 (s, 1H), 7.11 (dd, J=4.8, 1.8 Hz, 2H), 6.34 (tt, J=9.0, 2.3 Hz, 1H), 5.39 (d, J=9.0 Hz, 1H), 5.20 (s, 1H), 4.08 (dd, J=3.8, 2.3 Hz, 1H), 3.59 (dd, J=6.1, 4.3 Hz, 1H), 3.36 (t, J=11.9 Hz, 1H), 2.45-2.32 (m, 1H), 2.20-2.09 (m, 2H), 1.87-1.73 (m, 4H), 1.65-1.50 (m, 4H), 0.96 (d, J=6.0 Hz, 3H), 0.80 (d, J=8.1 Hz, 3H), 0.61 (d, J=7.1 Hz, 3H). ESI-MS m/z [M+H]⁺C₂₂H₂₉N₂F₂O₅:439.2.

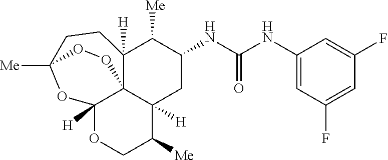 [SMILES: C[C@@H]1[C@H](NC(=O)Nc2cc(F)cc(F)c2)C[C@H]2[C@@H](C)CO[C@@H]3O[C@@]4(C)CC[C@@H]1[C@]32OO4]

### 1-(3,5-difluorophenyl)-3-((3R,5aS,6S,7R,8aS,9R,12R,12aR)-3,6,9-trimethyldecahydro-12H-3,12-epoxy[1,2]dioxepino[4,3-i]isochromen-7-yl) urea (PC-101)

¹H NMR (300 MHz, CDCl₃) § 8.04 (d, J=109.3 Hz, 1H), 7.03-6.88 (m, 2H), 6.39-6.26 (m, 1H), 5.56 (d, J=8.5 Hz, 1H), 5.16 (s, 1H), 3.75 (p, J=6.5 Hz, 2H), 3.70-3.61 (m, 1H), 3.38 (q, J=11.8 Hz, 2H), 2.64-2.44 (m, 1H), 2.40-2.21 (m, 2H), 2.02-1.81 (m, 3H), 1.74-1.66 (m, 1H), 1.44-1.38 (m, 1H), 1.36 (s, 3H), 1.29 (d, J=3.0 Hz, 1H), 0.98 (d, J=6.0 Hz, 3H), 0.70 (d, J=7.2 Hz, 3H). ESI-MS m/z [M+H]⁺C₂H₂₉N₂F₂O₅:439.2.

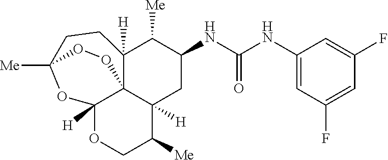 [SMILES: C[C@@H]1[C@@H](NC(=O)Nc2cc(F)cc(F)c2)C[C@H]2[C@@H](C)CO[C@@H]3O[C@@]4(C)CC[C@@H]1[C@]32OO4]

The disclosure of quantities and numerical values in this specification is not meant to be limiting.

We claim:

1. A method of treating malaria comprising:
administering a pharmaceutical composition comprising a compound, or
pharmaceutically acceptable salt thereof, comprising the following molecular structure:

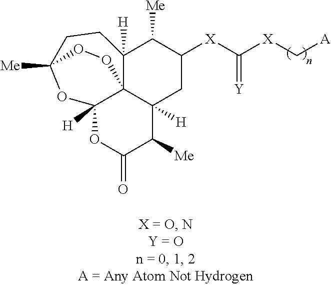

2. A method of treating malaria comprising:
administering a pharmaceutical composition comprising a compound, or a pharmaceutically acceptable salt thereof, comprising the following molecular structure:

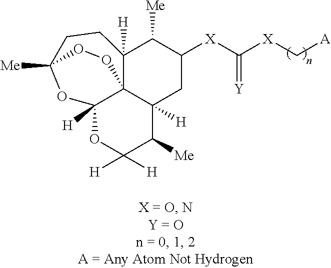

3. A pharmaceutical composition comprising the follow molecular structure:

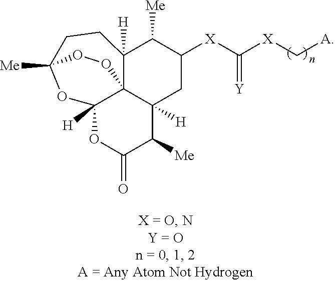

4. The pharmaceutical composition of claim 3 comprising the following molecular structure:

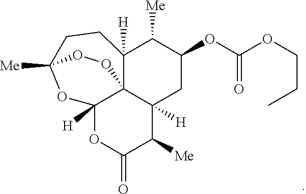 [SMILES: CCCOC(=O)O[C@H]1C[C@H]2[C@@H](C)C(=O)O[C@@H]3O[C@]4(C)CC[C@@H]([C@@H]1C)[C@]32OO4]

5. The pharmaceutical composition of claim 3 comprising the following molecular structure:

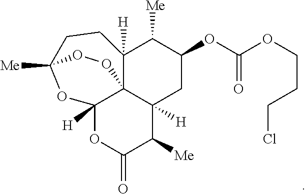 [SMILES: C[C@@H]1[C@@H](OC(=O)OCCCCl)C[C@H]2[C@@H](C)C(=O)O[C@@H]3O[C@]4(C)CC[C@@H]1[C@]32OO4]

6. A pharmaceutical composition comprising the following molecular structure:

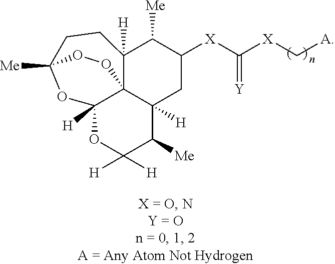

7. The pharmaceutical composition of claim 6 having the following molecular structure:

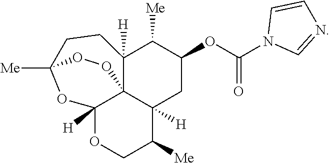 [SMILES: C[C@@H]1[C@@H](OC(=O)n2ccnc2)C[C@H]2[C@@H](C)CO[C@@H]3O[C@@]4(C)CC[C@@H]1[C@]32OO4]

8. The pharmaceutical composition of claim 6 having the following molecular structure:

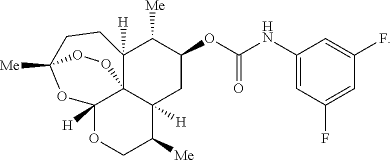 [SMILES: C[C@@H]1[C@@H](OC(=O)Nc2cc(F)cc(F)c2)C[C@H]2[C@@H](C)CO[C@@H]3O[C@@]4(C)CC[C@@H]1[C@]32OO4]

9. The pharmaceutical composition of claim 6 having the following molecular structure:

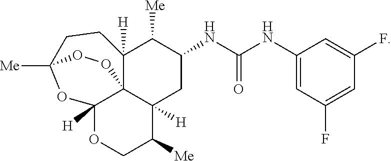 [SMILES: C[C@@H]1[C@H](NC(=O)Nc2cc(F)cc(F)c2)C[C@H]2[C@@H](C)CO[C@@H]3O[C@@]4(C)CC[C@@H]1[C@]32OO4]

10. The pharmaceutical composition of claim 6 having the following molecular structure:

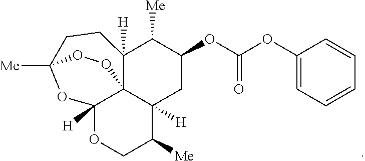 [SMILES: C[C@@H]1[C@@H](OC(=O)Oc2ccccc2)C[C@H]2[C@@H](C)CO[C@@H]3O[C@@]4(C)CC[C@@H]1[C@]32OO4]

11. The pharmaceutical composition of claim 6 having the following molecular structure:

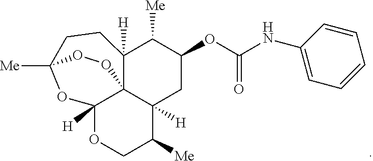 [SMILES: C[C@@H]1[C@@H](OC(=O)Nc2ccccc2)C[C@H]2[C@@H](C)CO[C@@H]3O[C@@]4(C)CC[C@@H]1[C@]32OO4]

12. The pharmaceutical composition of claim 6 having the following molecular structure:

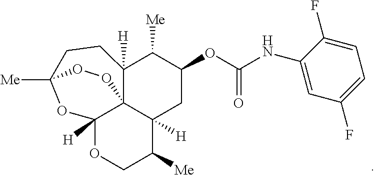 [SMILES: C[C@@H]1[C@@H](OC(=O)Nc2cc(F)ccc2F)C[C@H]2[C@@H](C)CO[C@@H]3O[C@@]4(C)CC[C@@H]1[C@]32OO4]

13. The pharmaceutical composition of claim 6 having the following molecular structure:

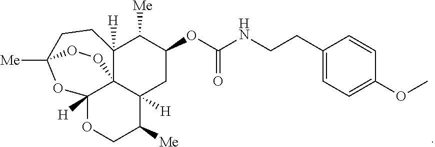 [SMILES: COc1ccc(CCNC(=O)O[C@H]2C[C@H]3[C@@H](C)CO[C@@H]4O[C@@]5(C)CC[C@@H]([C@@H]2C)[C@]43OO5)cc1]

14. The pharmaceutical composition of claim 6 having the following molecular structure:

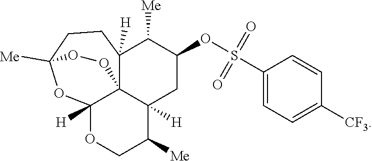 [SMILES: C[C@@H]1[C@@H](OS(=O)(=O)c2ccc(C(F)(F)F)cc2)C[C@H]2[C@@H](C)CO[C@@H]3O[C@@]4(C)CC[C@@H]1[C@]32OO4]

15. The pharmaceutical composition of claim 6 having the following molecular structure:

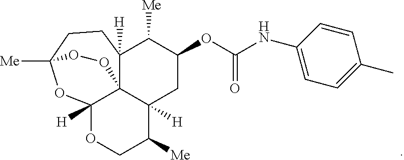 [SMILES: Cc1ccc(NC(=O)O[C@H]2C[C@H]3[C@@H](C)CO[C@@H]4O[C@@]5(C)CC[C@@H]([C@@H]2C)[C@]43OO5)cc1]

16. The pharmaceutical composition of claim 6 having the following molecular structure:

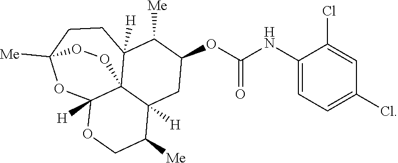 [SMILES: C[C@@H]1[C@@H](OC(=O)Nc2ccc(Cl)cc2Cl)C[C@H]2[C@@H](C)CO[C@@H]3O[C@@]4(C)CC[C@@H]1[C@]32OO4]

17. The pharmaceutical composition of claim 6 having the following molecular structure: 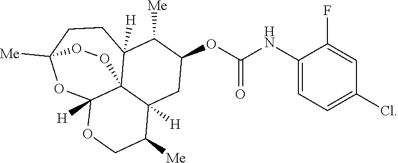 [SMILES: C[C@@H]1[C@@H](OC(=O)Nc2ccc(Cl)cc2F)C[C@H]2[C@@H](C)CO[C@@H]3O[C@@]4(C)CC[C@@H]1[C@]32OO4]
